(19) United States
(12) Patent Application Publication
(10) Pub. No.: US 2003/0009294 A1
(43) Pub. Date: Jan. 9, 2003

(54) Integrated system for gene expression analysis

(76) Inventor: Jill Cheng, Burlingame, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(21) Appl. No.: 10/026,110

(22) Filed: Dec. 20, 2001

Related U.S. Application Data
(60) Provisional application No. 60/297,210, filed on Jun. 7, 2001.

(51) Int. Cl. C12Q 1/68; G06F 19/00; G01N 33/48; G01N 33/50

(52) U.S. Cl. 702/20; 435/6

(57) ABSTRACT

In one embodiment of the invention, an integrated system is used to analyze gene expression data. The system integrates

### RELATED APPLICATION

This application claims the priority of U.S. Provisional Application No. 60/297,210, filed on Jun. 7, 2001. The '210 application is incorporated herein by reference for all purposes.

### BACKGROUND OF THE INVENTION

This invention is related to bioinformatics and biological data analysis. Specifically, the embodiments of the invention provides methods, computer software products and systems for gene expression analysis.

Biological assays using high density nucleic acid or protein probe arrays generate a large amount of data. Methods for storing, querying and analyzing such data have been disclosed in, for example, U.S. patent application Ser. Nos. 09/122,127, 09/122,169, and 09/122,304, all incorporated herein by reference in their entireties for all purposes.

While nucleic acid probe array technology has empowered us to generate huge amount of data, the analysis of these data has been challenging, especially the final step on associating biological significance with the experimental results. Typically, a microarray experiment generates several hundreds of potential hits. This may be too big a number to be validated by typical cell-based assays or animal experiments. Thus hits generated by statistical methods must be prioritized by biologists and only the top few will be pursued. Prioritization may require skilled biologist to sift through information about the hits, and then select the ones that ‘make most sense’ based on existing biological knowledge.

### SUMMARY OF THE INVENTION

In one aspect of the invention, methods for analyzing gene expression are provided. In some embodiments, the methods include the steps of obtaining expression levels of a plurality of genes; selecting at least one biological characteristic from a plurality of biological characteristics stored in a database; where the biological characteristics comprise genomic information about the genes, structural information about the products of the genes; and biological function of the genes; and analyzing the expression levels according to the selected at least one biological characteristic.

The analyzing may be grouping the expression levels according to the selected at least one biological characteristic. In some embodiments, the analyzing includes selecting the expression levels for further analysis according to the selected at least one biological characteristic. In some other embodiments, the analyzing includes clustering according to the selected at least one biological characteristic. Other analyzing steps may include multiple dimensional clustering according to selected biological characteristics and data mining.

The database may include information about orthologous genes, pathologic characteristics of genes (e.g., overexpression of a particular gene is related to a particular disease), splice variant information, protein domain information, signal pathway information, and/or gene ontology information. The database is typically a relational database, but it can also be other types of databases, such as an object-oriented database. For embodiments employing relational databases, SQL statements may be used to query the biological characteristic information.

In another aspect of the invention, a system for analyzing gene expression is provided. The system includes a processor; and a memory being coupled with the processor, the memory storing a plurality of machine instructions that cause the processor to perform the method steps of obtaining expression levels of a plurality of genes; selecting at least one biological characteristic from a plurality of biological characteristics stored in a database; where the biological characteristics comprise genomic information about the genes, structural information about the products of the genes; and biological function of the genes; and analyzing the expression levels according to the selected at least one biological characteristic.

The analyzing may be grouping the expression levels according to the selected at least one biological characteristic. In some embodiments, the analyzing includes selecting the expression levels for further analysis according to the selected at least one biological characteristic. In some other embodiments, the analyzing includes clustering according to selected at least one biological characteristic. Other analyzing steps may include multiple dimensional clustering according to selected biological characteristics and data mining.

The database may include information about orthologous genes, pathologic characteristics of genes (e.g., overexpression of a particular gene is related to a particular disease), splice variant information, protein domain information, signal pathway information, and/or gene ontology information. The database is typically a relational database, but it can also be other types of databases, such as an object-oriented database. For embodiments employing relational databases, SQL statements may be used to query the biological characteristic information.

In yet another aspect of the invention, a computer readable medium is provided. The computer readable medium contains computer-executable instructions for performing the methods comprising: obtaining expression levels of a plurality of genes; selecting at least one biological characteristic from a plurality of biological characteristics stored in a database; where the biological characteristics comprise genomic information about the genes, structural information about the products of the genes; and biological function of the genes; and analyzing the expression levels according to the selected at least one biological characteristic.

The analyzing may be grouping the expression levels according to the selected at least one biological characteristic. In some embodiments, the analyzing includes selecting the expression levels for further analysis according to the selected at least one biological characteristic. In some other embodiments, the analyzing includes clustering according to selected at least one biological characteristic. Other analyzing steps may include multiple dimensional clustering according to selected biological characteristics and data mining.

The database may include information about orthologous genes, pathologic characteristics of genes (e.g., overexpression of a particular gene is related to a particular disease), splice variant information, protein domain information, signal pathway information, and/or gene ontology information. The database is typically a relational database, but it can also be other types of databases, such as an object-oriented database. For embodiments employing relational databases, SQL statements may be used to query the biological characteristic information.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 illustrates an example of a computer system that may be utilized to execute the software of an embodiment of the invention.

FIG. 2 illustrates a system block diagram of the computer system of FIG. 1.

FIG. 3 shows exemplary multi-tier networked database architecture.

FIG. 4 shows a logical model for an exemplary biological characteristic database.

FIG. 5 is the physical model of the database of FIG. 4.

### DETAILED DESCRIPTION OF THE PREFERRED EMBODIMENTS

Reference will now be made in detail to the preferred embodiments of the invention. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention. All cited references, including patent and non-patent literature, are incorporated herein by reference in their entireties for all purposes.

I. Database Management Systems (DBMS)

In one aspect of the invention, methods, computer software, data structures and systems are provided for efficient data storage and retrieval. The embodiments of the invention employs DBMS for data storage and retrieval. The software products of the invention may be a part of a DBMS or interact with a DBMS. In addition, the data structure of the invention may reside in a DBMS.

A DBMS is a computerized record-keeping system that stores, maintains and provides access to information. For a general overview of the DBMS, see, e.g., Fred R. McFadden, et al, Modem Database Management, Oracle 7.3.4 edition, Hardcover (June 1999), Addison-Wesley Pub Co (Net); ISBN: 0805360549, which is incorporated herein by reference for all purposes. Commercial DBMSs are available from, for example, Oracle, Microsoft, and IBM.

A database system generally involves three major components: Data, Hardware and Software. Data itself consists of individual entities, in addition to which there will be relationships between entity types linking them together. The mapping of the collection of data onto a DBMS is usually done based on a data model. Various architectures exists for databases and various models have been proposed including the relational, network, and hierarchic models.

Conventional DBMS hardware consists of storage devices, typically, secondary storage devices, usually hard disks, on which the database physically resides, together with the associated I/O devices, device controllers, I/O channels and etc. Databases run on a range of machines, from personal computers to large mainframes, including database machines, which is hardware designed specifically to support a database system. For a description of basic computer systems and computer networks, see, e.g., Introduction to Computing Systems: From Bits and Gates to C and Beyond by Yale N. Patt, Sanjay J. Patel, 1st edition (Jan. 15, 2000) McGraw Hill Text; ISBN: 0072376902; and Introduction to Client/Server Systems: A Practical Guide for Systems Professionals by Paul E. Renaud, 2nd edition (June 1996), John Wiley & Sons; ISBN: 0471133337, both are incorporated herein by reference in their entireties for all purposes.

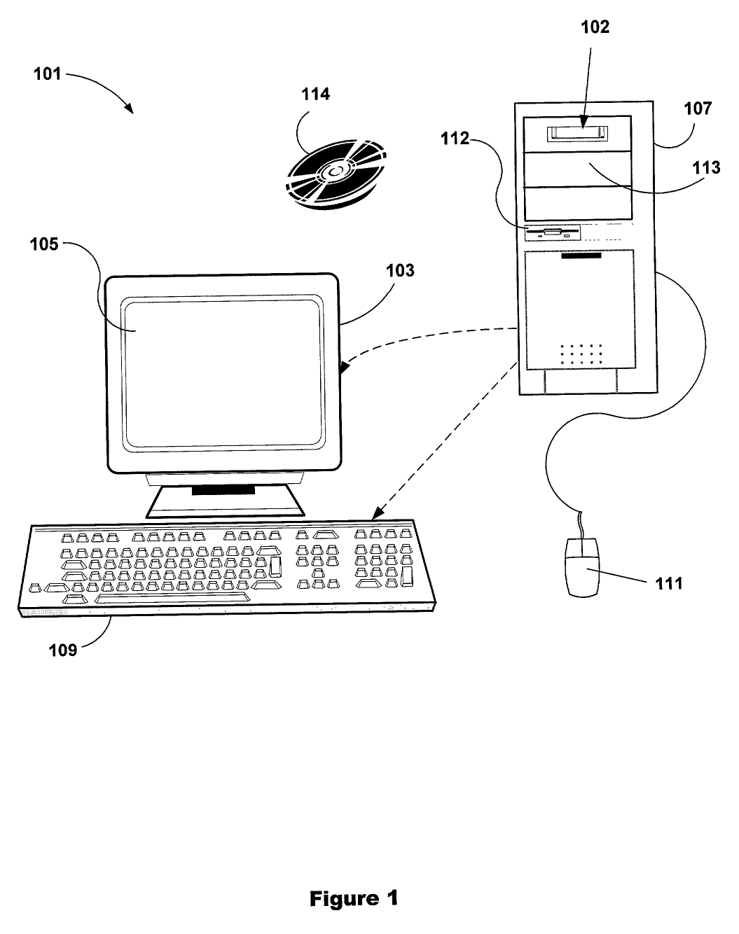

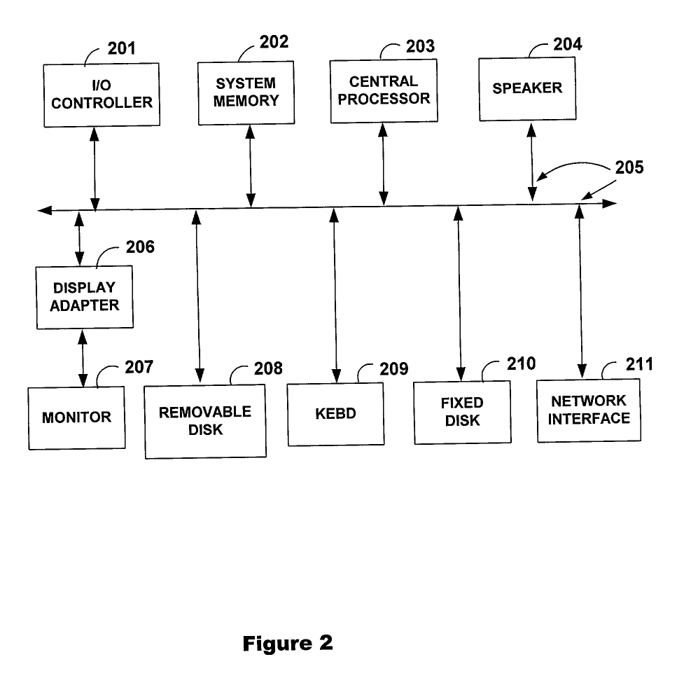

FIG. 1 illustrates an example of a computer system that may be used to execute the software of an embodiment of the invention, for storing data according to embodiments of the methods, software and systems of the invention. The computer system described herein is also suitable for hosting a DBMS. FIG. 1 shows a computer system 101 that includes a display 103, screen 105, cabinet 107, keyboard 109, and mouse 111. Mouse 111 may have one or more buttons for interacting with a graphic user interface. Cabinet 107 houses a floppy drive 112, CD-ROM or DVD-ROM drive 102, system memory and a hard drive (113) (see also FIG. 2) which may be utilized to store and retrieve software programs incorporating computer code that implements the invention, data for use with the invention and the like. Although a CD 114 is shown as an exemplary computer readable medium, other computer readable storage media including floppy disk, tape, flash memory, system memory, and hard drive may be utilized. Additionally, a data signal embodied in a carrier wave (e.g., in a network including the Internet) may be the computer readable storage medium.

FIG. 2 shows a system block diagram of computer system 101 used to execute the software of an embodiment of the invention. As in FIG. 1, computer system 101 includes monitor 201, and keyboard 209. Computer system 101 further includes subsystems such as a central processor 203 (such as a Pentium™ III processor from Intel), system memory 202, fixed storage 210 (e.g., hard drive), removable storage 208 (e.g., floppy or CD-ROM), display adapter 206, speakers 204, and network interface 211. Other computer systems suitable for use with the invention may include additional or fewer subsystems. For example, another computer system may include more than one processor 203 or a cache memory. Computer systems suitable for use with the invention may also be embedded in a measurement instrument.

When a DBMS runs on a computer, it typically runs as yet another application program. In between the DBMS and the hardware of the machine lies the host machine's operating system such as UNIX, Windows NT, Windows 2000, Linux or VAX/VMS, file manager and disk manager which deal with the file structure of the operating system and the page structure of the machine. DBMS may also run in a distributed fashion in several, even a large number of, machines connected via a network.

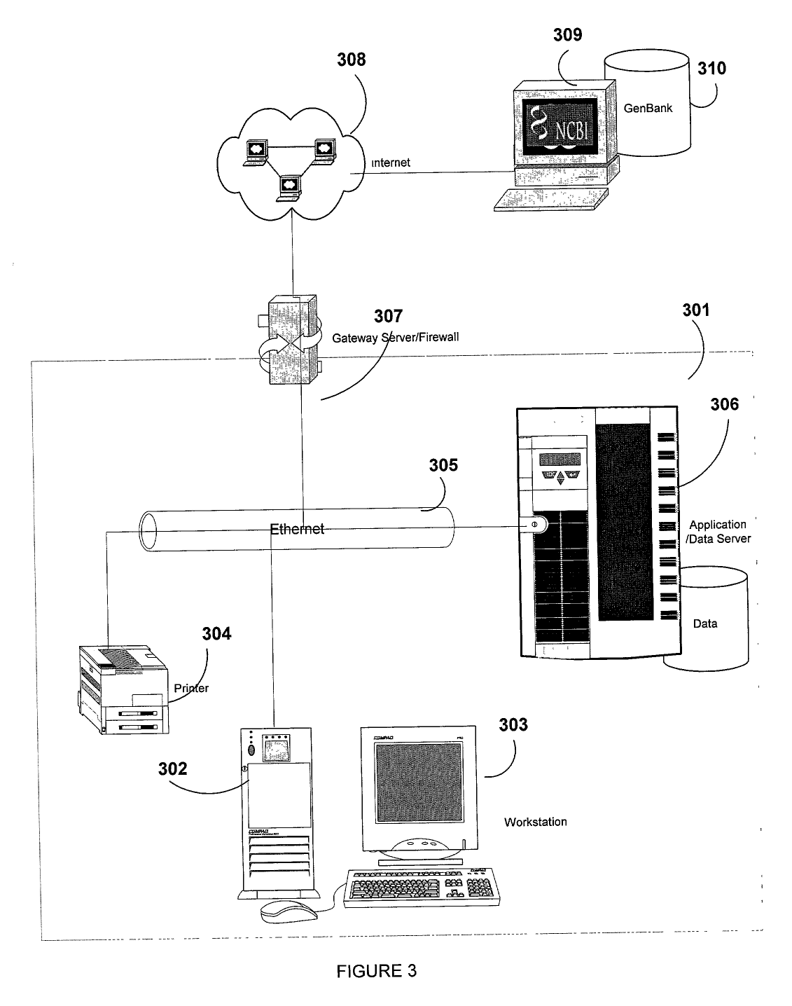

FIG. 3 shows an embodiment of a multi-tier internet database system that is useful for some embodiments of the invention (For a description of an Internet database platform, see, e.g., the Java™ 2 Platform, Enterprise Edition Application Programming Model described by Sun Microsystems, see http://java.sun.com/j2ee/apm/, last accessed on Dec. 14, 2000). The database (301), e.g, a gene expression database or a genotyping database, and system external to the data (302) reside in one or several data servers which constitute the data server tier.

Java enabled application servers (303) contain distributed, reusable business components housed in either a Java Common Object Request Broker Architecture (CORBA) Object Request Broker (ORB) or an Enterprise JavaBean (EJB) server. For a description of the distribute object technology, see, e.g., specifications and other documents at the web-site of the Object Management Group (OMG), http://www.omg.org, all incorporated herein by reference for all purposes.

The business components publish their data and services to Graphic User Interface (GUI) clients or other servers via component application programming interfaces (APIs) like CORBA and EJB, messaging APIs like Java Messenger Service (JMS), or data exchange formats like Extensible Markup Language (XML). The April 2000 specification of the XML is available at the http://www.w3.org and is incorporated herein by reference for all purposes.

The business components typically encapsulate and interact with persistent data stored within a standard relational database accessed via Java Database Connectivity (JDBC). Business components may also encapsulate data and services that are integrated from a variety of different data stores and applications.

Thin client HTML interfaces (305) are dynamically generated by Java enabled web servers (304) using, for example, JavaServer Pages (JSP) and Java Servlet standards (www.javasoft.com). More functionally rich and productive thick clients are assembled from libraries of reusable JavaBeans. The Java clients can run either as applets augmenting HTML within a Java enabled browser (306) or as applications running independently on the desktop (307). Java clients typically connect to application servers via Internet Inter-ORB Protocol (IIOP) or directly to data servers using JDBC.

II. Relational Database Model

Different models of data lead to different organizations. In general the relational model is preferred for storing probe array data in some embodiments.

Relational databases store all of their information in groups known as tables. Each database can contain one or more of these tables. A relational database management system (RDBMS) can also manage many individual underlying databases, with each one of these databases containing many tables. These tables are related to each other using some type of common element. A table can be thought of as containing a number of rows and columns. Each individual element stored in the table is known as a column. Each set of data within the table is known as a row. There are a number of commercial or public domain relational DBMS (RDBMS) such as Oracle (www.oracle.com), Sybase (www.sybase.com), Microsoft® SQL server and MYSQL (www.mvsql.com).

One preferred language for managing relational database is the SQL. Structured Query Language (SQL) is an American National Standard Institute (ANSI) standard computer programming language. SQL is useful for querying and managing relational databases. The ANSI standard for SQL (SQL-92, available at www.ansi.org, last visited on Dec. 14, 2000 and is incorporated herein by reference for all purposes) specifies a core syntax for the language itself For a detailed description of the SQL language, see, e.g., The Practical SQL Handbook: Using Structured Query Language by Judith S. Bowman, et al, Addison-Wesley Pub Co; ISBN: 0201447878, which is incorporated herein by reference for all purposes. Many embodiments of the invention employ SQL for query and database management.

One important process for designing a relational database is normalization. Normalization is the process of organizing data in a database. This includes creating tables and establishing relationships between those tables according to rules designed both to protect the data and to make the database more flexible by eliminating two factors: redundancy and inconsistent dependency. Redundant data waste disk space and creates maintenance problems. If data that exists in more than one place must be changed, the data must be changed in exactly the same way in all locations, which is inefficient and error prone. Inconsistent dependencies can make data difficult to access; the path to find the data may be missing or broken. There are a few rules for database normalization. Each rule is called a “normal form.” If the first rule is observed, the database is said to be in “first normal form.” If the first three rules are observed, the database is considered to be in “third normal form.” Although other levels of normalization are possible, third normal form is considered the highest level necessary for most applications. For a description of the normalization process, see, e.g, Handbook of Relational Database Design by Candace C. Fleming, et al. Addison-Wesley Pub Co; ISBN: 0201114348, which is incorporated herein by reference for all purposes.

Relational databases are an excellent way to organize data, but there can be a big per-row overhead in data storage and retrieval when there is a large number of rows in database tables. For example, in a fully normalized design, one row of data is reserved for every intensity value obtained in assays using high density probe arrays. Storing one row of data for every intensity value becomes less efficient in some systems when there are thousands of scans and billions of values.

In one aspect of the invention, methods, systems, data structures and computer software are provided to efficiently store and retrieve intensity data. The methods, systems, data structures and computer software are also useful for processing of any other large dataset.

III. High Density Probe Arrays

The methods of the invention are particularly useful for storing probe intensity data generated using high density probe arrays, such as high density nucleic acid probe arrays. High density nucleic acid probe arrays, also referred to as “DNA Microarrays,” have become a method of choice for monitoring the expression of a large number of genes and for detecting sequence variations, mutations and polymorphisms. As used herein, “Nucleic acids” may include any polymer or oligomer of nucleosides or nucleotides (polynucleotides or oligonucleotidies), which include pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982) and L. Stryer BIOCHEMISTRY, 4^{th} Ed., (March 1995), both incorporated by reference. “Nucleic acids” may include any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

“A target molecule” refers to a biological molecule of interest. The biological molecule of interest can be a ligand, receptor, peptide, nucleic acid (oligonucleotide or polynucleotide of RNA or DNA), or any other of the biological molecules listed in U.S. Pat. No. 5,445,934 at col. 5, line 66 to col. 7, line 51. For example, if transcripts of genes are the interest of an experiment, the target molecules would be the transcripts. Other examples include protein fragments, small molecules, etc. “Target nucleic acid” refers to a nucleic acid (often derived from a biological sample) of interest. Frequently, a target molecule is detected using one or more probes. As used herein, a “probe” is a molecule for detecting a target molecule. It can be any of the molecules in the same classes as the target referred to above. A probe may refer to a nucleic acid, such as an oligonucleotide, capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, any ligands for detecting its binding partners. When referring to targets or probes as nucleic acids, it should be understood that these are illustrative embodiments that are not to limit the invention in any way.

In preferred embodiments, probes may be immobilized on substrates to create an array. An “array” may comprise a solid support with peptide or nucleic acid or other molecular probes attached to the support. Arrays typically comprise a plurality of different nucleic acids or peptide probes that are coupled to a surface of a substrate in different, known locations. These arrays, also described as “microarrays” or colloquially “chips” have been generally described in the art, for example, in Fodor et al., Science, 251:767-777 (1991), which is incorporated by reference for all purposes. Methods of forming high density arrays of oligonucleotides, peptides and other polymer sequences with a minimal number of synthetic steps are disclosed in, for example, U.S. Pat. Nos. 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes. The oligonucleotide analogue array can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668, U.S. Pat. Nos. 5,677,195, 5,800,992 and 6,156,501 which disclose methods of forming vast arrays of peptides, oligonucleotides and other molecules using, for example, light-directed synthesis techniques. See also, Fodor et al., Science, 251, 767-77 (1991). These procedures for synthesis of polymer arrays are now referred to as VLSIPS™ procedures. Using the VLSIPS™ approach, one heterogeneous array of polymers is converted, through simultaneous coupling at a number of reaction sites, into a different heterogeneous array. See, U.S. Pat. Nos. 5,384,261 and 5,677,195.

Methods for making and using molecular probe arrays, particularly nucleic acid probe arrays are also disclosed in, for example, U.S. Pat. Nos. 5,143,854, 5,242,974, 5,252,743, 5,324,633, 5,384,261, 5,405,783, 5,409,810, 5,412,087, 5,424,186, 5,429,807, 5,445,934, 5,451,683, 5,482,867, 5,489,678, 5,491,074, 5,510,270, 5,527,681, 5,527,681, 5,541,061, 5,550,215, 5,554,501, 5,556,752, 5,556,961, 5,571,639, 5,583,211, 5,593,839, 5,599,695, 5,607,832, 5,624,711, 5,677,195, 5,744,101, 5,744,305, 5,753,788, 5,770,456, 5,770,722, 5,831,070, 5,856,101, 5,885,837, 5,889,165, 5,919,523, 5,922,591, 5,925,517, 5,658,734, 6,022,963, 6,150,147, 6,147,205, 6,153,743, 6,140,044 and D430024, all of which are incorporated by reference in their entireties for all purposes.

Typically, a nucleic acid sample is labeled with a signal moiety, such as a fluorescent label. The sample is hybridized with the array under appropriate conditions. The arrays are washed or otherwise processed to remove non-hybridized sample nucleic acids. The hybridization is then evaluated by detecting the distribution of the label on the chip. The distribution of label may be detected by scanning the arrays to determine fluorescence intensity distribution. Typically, the hybridization of each probe is reflected by several pixel intensities. The raw intensity data may be stored in a gray scale pixel intensity file. The GATC™ Consortium has specified several file formats for storing array intensity data. The final software specification is available at www.gatcconsortium.org and is incorporated herein by reference in its entirety. The pixel intensity files are usually large. For example, a GATC™ compatible image file may be approximately 50 Mb if there are about 5000 pixels on each of the horizontal and vertical axes and if a two byte integer is used for every pixel intensity. The pixels may be grouped into cells (see, GATC™ software specification). The probes in a cell are designed to have the same sequence (i.e., each cell is a probe area). A CEL file contains the statistics of a cell, e.g., the 75th percentile and standard deviation of intensities of pixels in a cell. The 50, 60, 70, 75 or 80th percentile of pixel intensity of a cell is often used as the intensity of the cell.

Methods for signal detection and processing of intensity data are additionally disclosed in, for example, U.S. Pat. Nos. 5,547,839, 5,578,832, 5,631,734, 5,800,992, 5,856,092, 5,936,324, 5,981,956, 6,025,601, 6,090,555, 6,141,096, 6,141,096, and 5,902,723. Methods for array based assays, computer software for data analysis and applications are additionally disclosed in, e.g., U.S. Pat. Nos. 5,527,670, 5,527,676, 5,545,531, 5,622,829, 5,631,128, 5,639,423, 5,646,039, 5,650,268, 5,654,155, 5,674,742, 5,710,000, 5,733,729, 5,795,716, 5,814,450, 5,821,328, 5,824,477, 5,834,252, 5,834,758, 5,837,832, 5,843,655, 5,856,086, 5,856,104, 5,856,174, 5,858,659, 5,861,242, 5,869,244, 5,871,928, 5,874,219, 5,902,723, 5,925,525, 5,928,905, 5,935,793, 5,945,334, 5,959,098, 5,968,730, 5,968,740, 5,974,164, 5,981,174, 5,981,185, 5,985,651, 6,013,440, 6,013,449, 6,020,135, 6,027,880, 6,027,894, 6,033,850, 6,033,860, 6,037,124, 6,040,138, 6,040,193, 6,043,080, 6,045,996, 6,050,719, 6,066,454, 6,083,697, 6,114,116, 6,114,122, 6,121,048, 6,124,102, 6,130,046, 6,132,580, 6,132,996 and 6,136,269, all of which are incorporated by reference in their entireties for all purposes.

IV. Integration of Biological Knowledge in Gene Expression Analysis

Nucleic acid probe array technology has revolutionized the way biological activities of cells like growth, drug response, and diseases are examined. Expression of thousands of genes can be monitored simultaneously with a minute amount of material. For the first time, genes can be analyzed in the context of all genes that might work in concert in directing biological processes. While this technology has empowered scientists to generate huge amount of data, the analysis of these data has been challenging, especially the final step on associating biological significance with the experimental results.

In one aspect of the invneiton, a relational data model is designed for the integration of biological knowledge with expression data. Biological knowledge is integrated following the central dogma of biological macromolecules: DNA, mRNA and protein. Database entities were designed to mimic the biological entities, the relationship among entities mimics the relationship among biological macromolecules, for instance, one gene can have many orthologous loci, one locus can produces many transcripts, and one transcript can generate one or more proteins. This data model is also faithful to the way biological knowledge is organized. For example, a protein domain is linked to protein entity because it's a property of protein, gene ontology is associated with the locus entity because it's knowledge developed against a DNA locus.

Using this database, biological knowledge is transformed and can be represented by symbolic handles (e.g., a primary key to a row of a datatable, a row ID, etc). This approach allows one with incomplete knowledge about the genes understudy to perform a relatively through analysis of gene expression data. For example, building a knowledge metrics for microarray data analysis, or do biological clustering of genes. Statistical methods in current analysis pipeline may be applied only to groups of genes with certain characteristics, this will help reducing the noise and thus increase the sensitivity. Also, clusters generated from statistical methods can be evaluated by analyzing the biological relevance against the database, this will help evaluating different statistical methods and thus assists performance tuning. Since knowledge can be represented by handles, and can be analyzed in batch by computer, the manual effort will be minimized. The ‘making sense’ of potential hits can be done efficiently and accurately.

Knowledge regarding orthologous genes, pathology, splice variants, protein domains, signaling pathways, and gene ontology are integrated with expression data. Gene ontology provides a simple way to classify genes based on existing knowledge; it can be used to measure the biological distance between genes. Several database tables are designed to represent the direct acyclic graph (DAG) structure of ontology. Several tables are designed to resolve all possible paths to facilitate the measurement of distances between genes. This database may serve as the biological platform for microarray data analysis.

In one aspect of the invention, methods for analyzing gene expression are provided. In some embodiments, the methods include the steps of obtaining expression levels of a plurality of genes; selecting at least one biological characteristic from a plurality of biological characteristics stored in a database; where the biological characteristics comprise genomic information about the genes, structural information about the products of the genes; and biological function of the genes; and analyzing the expression levels according to the selected at least one biological characteristic. The expression levels can be relative or absolute levels of any measurements that can indicate the expression of genes. For example, the expression levels can be RNA transcript concentrations (micromolar or other units) in a sample; RNA transcript concentrations relative to a particular transcript; protein concentrations in sample etc. One of skill in the art would appreciate that the invention is not limited to any particular measurement of gene expresion or any particular technology for measuring gene expression. However, many embodiments of the invention are particularly suitable for analyzing the expression of a large number of, at least 50, 100, 500, 1000, 5000 and 10,000 genes. The term “biological characteristic,” as used herein, refers broadly to any characteristics that has biological relevancy. For example, a biological characteristic may be chromosomal location, cellular location (particularly for intermediate or final products of gene expression), molecular or cellular functions, structural information (including sequence information, three dimensional structure, protein domains, etc.). In one embodiments, the biological characterstics are described using gene ontology system. The Gene Ontology Consortium (GO) provides a set of standardized vocabulary to describe various biological characteristics. The three organizing principles of GO are molecular function, biological process and cellular component. The current gene ontology information is available at the Gene Ontology Consortum web site at (www.geneontology.com).

The analyzing may be grouping the expression levels according to the selected at least one biological characteristic. For example, genes may be grouped according to their role in a regulatory pathway. In some embodiments, the analyzing includes selecting the expression levels for further analysis according to the selected at least one biological characteristic. For example, genes that are known to be involved in the immune system may be selected for cluster analysis. In some other embodiments, the analyzing includes clustering according to selected at least one biological characteristic. Other analyzing steps may include multiple dimensional clustering according to selected biological characteristics and data mining.

The database may include information about orthologous genes, pathologic characteristics of genes (e.g., overexpression of a particular gene is related to a particular disease), splice variant information, protein domain information, signal pathway information, and/or gene ontology information. The database is typically a relational database, but it can also be other types of databases, such as an object-oriented database. For embodiments employing relational databases, SQL statements may be used to query the biological characteristic information.

In another aspect of the invention, a system for analyzing gene expression is provided. The system includes a processor; and a memory being coupled with the processor, the memory storing a plurality of machine instructions that cause the processor to perform the method steps of obtaining expression levels of a plurality of genes; selecting at least one biological characteristic from a plurality of biological characteristics stored in a database; where the biological characteristics comprise genomic information about the genes, structural information about the products of the genes; and biological function of the genes; and analyzing the expression levels according to the selected at least one biological characteristic.

The analyzing may be grouping the expression levels according to the selected at least one biological characteristic. In some embodiments, the analyzing includes selecting the expression levels for further analysis according to the selected at least one biological characteristic. In some other embodiments, the analyzing includes clustering according to selected at least one biological characteristic. Other analyzing steps may include multiple dimensional clustering according to selected biological characteristics and data mining.

The database may include information about orthologous genes, pathologic characteristics of genes (e.g., overexpression of a particular gene is related to a particular disease), splice variant information, protein domain information, signal pathway information, and/or gene ontology information. The database is typically a relational database, but it can also be other types of databases, such as an object-oriented database. For embodiments employing relational databases, SQL statements may be used to query the biological characteristic information.

In yet another aspect of the invention, a computer readable medium is provided. The computer readable medium contains computer-executable instructions for performing the methods comprising: obtaining expression levels of a plurality of genes; selecting at least one biological characteristic from a plurality of biological characteristics stored in a database; where the biological characteristics comprise genomic information about the genes, structural information about the products of the genes; and biological function of the genes; and analyzing the expression levels according to the selected at least one biological characteristic.

The analyzing may be grouping the expression levels according to the selected at least one biological characteristic. In some embodiments, the analyzing includes selecting the expression levels for further analysis according to the selected at least one biological characteristic. In some other embodiments, the analyzing includes clustering according to selected at least one biological characteristic. Other analyzing steps may include multiple dimensional clustering according to selected biological characteristics and data mining.

The database may include information about orthologous genes, pathologic characteristics of genes (e.g., overexpression of a particular gene is related to a particular disease), splice variant information, protein domain information, signal pathway information, and/or gene ontology information. The database is typically a relational database, but it can also be other types of databases, such as an object-oriented database. For embodiments employing relational databases, SQL statements may be used to query the biological characteristic information.

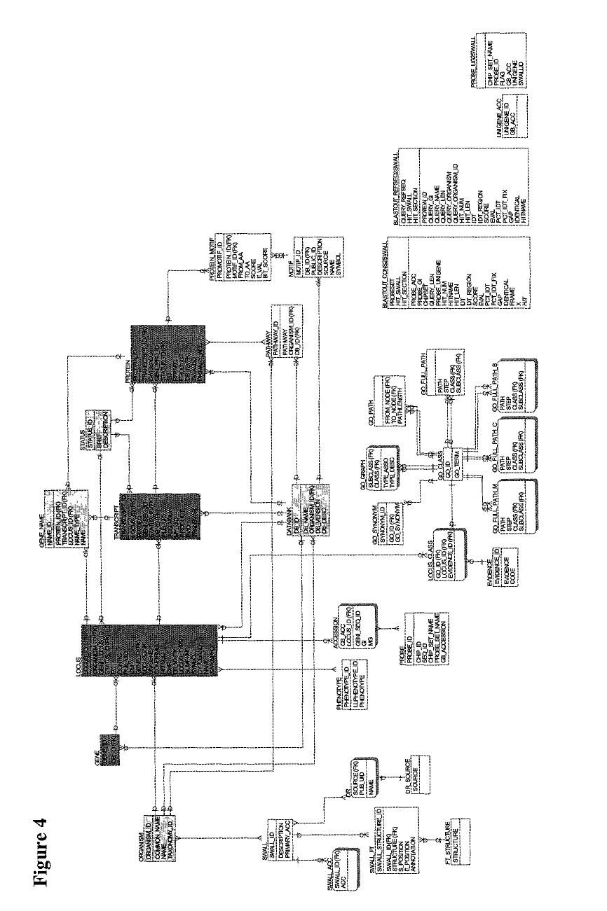

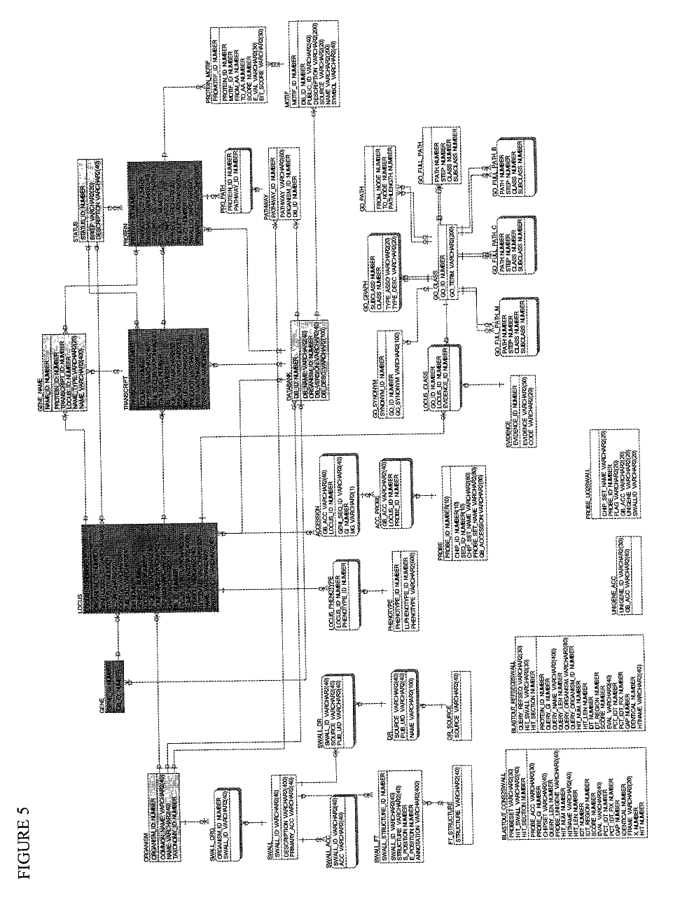

FIGS. 4 and 5 shows an exemplary relational database for managing biological characteristic information. The database was designed using Erwin and the database was implemented in Oracle 8.0i. Biological information was downloaded from public domain and was processed using Per1 scripts.

In this exemplary embodiment, biological knowledge is integrated following the central dogma of biological macromolecules: DNA, mRNA and protein. Database entities were designed to mimic the biological entities, the relationship among entities mimics the relationship among biological macromolecules, for instance, one gene can have many orthologous locus, one locus can produces many transcripts, and one transcript can generate one or more proteins. This data model is also faithful to the way biological knowledge is organized, thus driven by business rules. For example, protein domain is linked to protein entity because it's a property of protein, gene ontology is associate with the locus entity because it's knowledge developed against DNA locus.

The database also includes several reference tables:

1. Blastout_refseq2swall: blastx results of entire refseq against Swall (Swissprot+TrEMBL)

2. Blastout_cons2swall: blastx result of U95 consensus sequences against Swall

3. Blastout_unigene2swall: blastx results of U95 Unigene unique representative sequences against Swall

4. Unigene_acc: Human only, gb_acc in each Unigene cluster

5. Probe_ug2swall: another way to link probeset with Swall, all GB accessions from the same unigene cluster as the probesets are searched against the EMBL-reference in Swall, this table contains the hits.

Because the database is relational, SQL statements may be used to query the database. For example, the following SQL statements may be used to select all protein annotations for certain probesets from swiss+Tremb1:

select probe_set_name,swall_id,structure,s_position,e_position, annotation

from probe, probe_ug2swall, swall_ft

where probe_set_name in (‘34995_at’, ‘40214_at’)

and probe.probe_id=probe_ug2swall.probe_id

and probe_ug2swall.swallid=swall_ft.swall_id

The following is an output of the above instructions:

| | | | | | |
|---|---|---|---|---|---|
| 40214_at | CEGT_HUMAN | TRANSMEM | 11 | 31 | SIGNAL-ANCHOR (POTENTIAL) |
| 40214_at | CEGT_HUMAN | TRANSMEM | 286 | 306 | POTENTIAL |
| 40214_at | CEGT_HUMAN | TRANSMEM | 314 | 334 | POTENTIAL |
| 40214_at | CEGT_HUMAN | DOMAIN | 1 | 10 | LUMENAL (POTENTIAL) |
| 34995_at | CGRR_HUMAN | CHAIN | 23 | 461 | CALCITONIN GENE-RELATED PEPTIDE TYPE 1RECEPTOR |
| 34995_at | CGRR_HUMAN | TRANSMEM | 147 | 166 | 1 (POTENTIAL) |
| 34995_at | CGRR_HUMAN | TRANSMEM | 174 | 193 | 2 (POTENTIAL) |
| 34995_at | CGRR_HUMAN | TRANSMEM | 214 | 236 | 3 (POTENTIAL) |
| 34995_at | CGRR_HUMAN | TRANSMEM | 254 | 273 | 4 (POTENTIAL) |
| 34995_at | CGRR_HUMAN | TRANSMEM | 290 | 313 | 5 (POTENTIAL) |
| 34995_at | CGRR_HUMAN | TRANSMEM | 337 | 354 | 6 (POTENTIAL) |
| 34995_at | CGRR_HUMAN | TRANSMEM | 367 | 388 | 7 (POTENTIAL) |
| 34995_at | CGRR_HUMAN | DOMAIN | 23 | 146 | EXTRACELLULAR (POTENTIAL) |
| 34995_at | CGRR_HUMAN | DOMAIN | 167 | 173 | CYTOPLASMIC (POTENTIAL) |
| 34995_at | CGRR_HUMAN | DOMAIN | 194 | 213 | EXTRACELLULAR (POTENTIAL) |
| 34995_at | CGRR_HUMAN | DOMAIN | 237 | 253 | CYTOPLASMIC (POTENTIAL) |
| 34995_at | CGRR_HUMAN | DOMAIN | 274 | 289 | EXTRACELLULAR (POTENTIAL) |
| 34995_at | CGRR_HUMAN | DOMAIN | 314 | 336 | CYTOPLASMIC (POTENTIAL) |
| 34995_at | CGRR_HUMAN | DOMAIN | 355 | 366 | EXTRACELLULAR (POTENTIAL) |
| 34995_at | CGRR_HUMAN | DOMAIN | 389 | 461 | CYTOPLASMIC (POTENTIAL) |
| 34995_at | CGRR_HUMAN | CARBOHYD | 66 | 66 | N-LINKED (GLCNAC . . . ) (POTENTIAL) |
| 34995_at | CGRR_HUMAN | CARBOHYD | 118 | 118 | N-LINKED (GLCNAC . . . ) (POTENTIAL) |
| 34995_at | CGRR_HUMAN | CARBOHYD | 123 | 123 | N-LINKED (GLCNAC . . . ) (POTENTIAL) |
| 34995_at | CGRR_HUMAN | SIGNAL | 1 | 22 | POTENTIAL |

The following exemplary SQL statements may be used to find all U95 probe sets at the GeneChip® U95 probe array (available from Affymetrix, Inc., Santa Clara, Calif.) that has GO annotation related to ‘growth’

select distinct probe_set_name from probe, acc_probe

where chip_set_name like ‘%U95%’ and probe.probe_id=acc_probe.probe_id

and acc_probe.locus_id in (

select distinct locus_id

from go_class, locus_class where go_term like ‘%growth %’

and locus_class.go_id=go_class.go_id)

The following SQL statements may be used to find pfam domains that occur on genes with annotations related to ‘growth’

select distinct motif.name from motif, protein_motif, protein, transcript

where transcript.locus_id in (

select distinct locus_class.locus_id from locus_class, go_class

where motif.db_id=6

and go term like ‘%growth %’ and locus_class.go_id=go_class.go_id)

and transcript.transcript_id=protein.transcript_id

and protein.protein_id=protein_motif.protein_id

and protein_motif.motif_id=motif.motif_id

### CONCLUSION

It is to be understood that the above description is intended to be illustrative and not restrictive. For example, many embodiments are described using nucleic acid probe array as examples, one of skill in the art would appreciate that the methods, software and system of the inveniton can also be used to analyze other biological assays, including data from protein/peptide array experiments, and in general, data from any parallel assay systems. Many variations of the invention will be apparent to those of skill in the art upon reviewing the above description. The scope of the invention should, therefore, be determined not with reference to the above description, but should instead be determined with reference to the appended claims, along with the full scope of equivalents to which such claims are entitled.

All cited references, including patent and non-patent literature, are incorporated herein by reference in their entireties for all purposes.

What is claimed is:

1. A method for analyzing gene expression comprising:
obtaining expression levels of a plurality of genes;
selecting at least one biological characteristic from a plurality of biological characteristics stored in a database; wherein the biological characteristics comprise genomic information about the genes, structural information about the products of the genes; and biological function of the genes; and
analyzing the expression levels according to the selected at least one biological characteristic.

2. The method of claim 1 wherein the analyzing comprises grouping the expression levels according to the selected at least one biological characteristic.

3. The method of claim 1 wherein the analyzing comprises selecting the expression levels for further analysis according to the selected at least one biological characteristic.

4. The method of claim 1 wherein the analyzing comprises clustering according to selected at least one biological characteristic.

5. The method of claim 4 wherein the analyzing comprises multiple dimensional clustering according to selected biological characteristics.

6. The method of claim 6 wherein the analyzing comprises data mining.

7. The method of claim 1 wherein the plurality of biological characteristics comprise orthologous genes.

8. The method of claim 1 wherein the plurality of biological characteristics comprise pathologic characteristics of genes.

9. The method of claim 1 wherein the plurality of biological characteristics comprise splice variant information.

10. The method of claim 1 wherein the plurality of biological characteristics comprise protein domain information.

11. The method of claim 1 wherein the plurality of biological characteristics comprise signal pathway information.

12. The method of claim 1 wherein the plurality of biological characteristics comprise gene ontology information.

13. The method of claim 1 wherein the database is a relational database.

14. The method of claim 1 wherein the database is an object oriented database.

15. The method of claim 13 wherein the biological characteristics are retrived using SQL statements.

16. A system for analyzing gene expression comprising a processor; and a memory being coupled with the processor, the memory storing a plurality of machine instructions that cause the processor to perform the method steps of obtaining expression levels of a plurality of genes;
selecting at least one biological characteristic from a plurality of biological characteristics stored in a database; wherein the biological characteristics comprise genomic information about the genes, structural information about the products of the genes; and biological function of the genes; and
analyzing the expression levels according to the selected at least one biological characteristic.

17. The system of claim 16 wherein the analyzing comprises grouping the expression levels according to the selected at least one biological characteristic.

18. The system of claim 16 wherein the analyzing comprises selecting the expression levels for further analysis according to the selected at least one biological characteristic.

19. The system of claim 16 wherein the analyzing comprises clustering according to selected at least one biological characteristic.

20. The system of claim 16 wherein the analyzing comprises multiple dimensional clustering according to selected biological characteristics.

21. The system of claim 16 wherein the analyzing comprises data mining.

22. The system of claim 16 wherein the plurality of biological characteristics comprise orthologous genes.

23. The system of claim 16 wherein the plurality of biological characteristics comprise pathologic characteristics of genes.

24. The system of claim 16 wherein the plurality of biological characteristics comprise splice variant information.

25. The system of claim 16 wherein the plurality of biological characteristics comprise protein domain information.

26. The system of claim 16 wherein the plurality of biological characteristics comprise signal pathway information.

27. The system of claim 16 wherein the plurality of biological characteristics comprise gene ontology information.

28. The system of claim 16 wherein the database is a relational database.

29. The system of claim 16 wherein the database is an object oriented database.

30. The system of claim 28 wherein the biological characteristics are retrived using SQL statements.

31. A computer readable medium comprising computer-executable instructions for performing the methods comprising:
obtaining expression levels of a plurality of genes;
selecting at least one biological characteristic from a plurality of biological characteristics stored in a database; wherein the biological characteristics comprise genomic information about the genes, structural information about the products of the genes; and biological function of the genes; and
analyzing the expression levels according to the selected at least one biological characteristic.

32. The computer readable medium of claim 31 wherein the analyzing comprises grouping the expression levels according to the selected at least one biological characteristic.

33. The computer readable medium of claim 31 wherein the analyzing comprises selecting the expression levels for further analysis according to the selected at least one biological characteristic.

34. The computer readable medium of claim 31 wherein the analyzing comprises clustering according to selected at least one biological characteristic.

35. The computer readable medium of claim 31 wherein the analyzing comprises multiple dimensional clustering according to selected biological characteristics.

36. The computer readable medium of claim 31 wherein the analyzing comprises data mining.

37. The computer readable medium of claim 31 wherein the plurality of biological characteristics comprise orthologous genes.

38. The computer readable medium of claim 31 wherein the plurality of biological characteristics comprise pathologic characteristics of genes.

39. The computer readable medium of claim 31 wherein the plurality of biological characteristics comprise splice variant information.

40. The computer readable medium of claim 31 wherein the plurality of biological characteristics comprise protein domain information.

41. The computer readable medium of claim 31 wherein the plurality of biological characteristics comprise signal pathway information.

42. The computer readable medium of claim 31 wherein the plurality of biological characteristics comprise gene ontology information.

43. The computer readable medium of claim 31 wherein the database is a relational database.

44. The computer readable medium of claim 31 wherein the database is an object oriented database.

45. The computer readable medium of claim 43 wherein the biological characteristics are retrived using SQL statements.
